(19) United States
(12) Patent Application Publication
(10) Pub. No.: US 2024/0307458 A1
(43) Pub. Date: Sep. 19, 2024

(54) MEDICINE COMPOSITION FOR TREATMENT OF BONE AND JOINT DISEASES ORIGINATED FROM MEDICINAL MATERIALS AND THE PROCESS FOR PRODUCING THEREOF

(71) Applicant: SAO THAI DUONG JOINT STOCK COMPANY, Hanoi (VN)

(72) Inventor: Thi Huong Lien NGUYEN, Hanoi (VN)

(21) Appl. No.: 18/572,711

(22) PCT Filed: Jun. 21, 2022
(86) PCT No.: PCT/VN2022/000004

§ 371 Date: Dec. 20, 2023

(30) Foreign Application Priority Data
Jun. 21, 2021 (VN) 1-2021-03707

(51) Int. Cl. A61K 35/586; A61K 31/192; A61K 35/32; A61K 36/076; A61K 36/258; A61K 36/484; A61K 36/54; A61K 36/736; A61K 36/76; A61K 38/48; A61P 19/02

(52) U.S. Cl. CPC A61K 35/586; A61K 31/192; A61K 35/32; A61K 36/076; A61K 36/258; A61K 36/484; A61K 36/54; A61K 36/736; A61K 36/76; A61K 38/482; A61P 19/02; A61K 2236/13; A61K 2236/331; A61K 2236/37; A61K 2236/39; A61K 2236/51

(57) ABSTRACT

The present invention relates to a medicine composition for treatment of bone and joint diseases that has its ingredients (% by weight) as the dried extract of medicinal herbs: 40-75; tortoiseshell extract: 0.1-20; mixed bone extract: 0.1-20; deer antler extract: 0.1-20; salix alba extract: 5-25; nattokinase: 0.1-10; and Na-benzoate: 0-0.5, wherein the herbal medicine mixture comprises the ingredients of: Huang Bai (bark), Liquorice (root/rhizome), Hardy rubber tree (bark), Ox Knee (root), Fang Feng (root), Poria cocos wolf (fruit body), Chinese foxglove (rhizome), Sang Ji Sheng (whole tree), Gentian Root (root), Rhizoma Anemarrhena (rhizome), Peach blossom (blossom), Dried tangerine peel (peel), Chinese peony (root), Dangshen (root), Ginseng (root) Du Huo (root), Dongquai (root), Cinnamon (bough), Xi Xin (root, rhizome), Szechuan lovage (rhizome) and Dwarf umbrella tree (root). The present invention also relates to a process for producing medicine composition for treatment of bone and joint diseases mentioned above.

### FIELD OF THE INVENTION

The present invention relates to a field of pharmaceuticals. In particular, the present invention relates to a medicine composition for treatment of bone and joint diseases that has its ingredients originated from medicinal herbs in combination with animal bone extracts and the process for producing thereof.

### BACKGROUND ART

Bone and joint diseases, including osteoarthritis, is a common disease, clinically in both men and women. In Vietnam, on average, for every 1 million people, about 700 people have suffered osteoarthritis (0.0007%). The disease is common in the age of 15 and older, about 80% of patients have arthritis in their middle age.

Osteoarthritis, if not treated well, can cause dangerous complications for patients such as loss of mobility, labor, disability, increased risk of cardiovascular diseases, etc. There are about 10-15% of the osteoarthritis patients who face the risk of becoming disabled, unable to live on their own, but have to rely on the help of others. Therefore, persons with osteoarthritis pains need to be treated as soon as possible, comprehensively and closely monitored. However, the treatment of osteoarthritis pains is often difficult because the disease progresses are relatively quick, easily become complications and difficult to be treated completely.

According to Traditional Chinese Medicine (TCM), osteoarthritis pain is classified in the category of the Rat, i.e. pains caused by blocked blood vessels, poor blood circulation, kidney failure, weakened defenses of the body. These cause disturbances in blood circulation, obstructing the flow of blood to nourish cartilage, leading to pain, inflammation and swelling in joints.

Therefore, in order to treat osteoarthritis pain, TCM focuses on using combination medicines for eliminating rheumatism, heat-clearing, detoxification, clearing the meridians, nourishing the five organs, and improving blood circulation to enhance qi and blood flows, balance yin and yang.

There are many remedies for the treatment of bone and joint diseases. Below are some typical remedies made from herbs.

### Quyen Ty Thang Remedy

Ingredients: Qiang Huo, Du Huo, Cinnamon, Chinese peony, Liquorice, Fang Feng, Ginger, Osmanthus Fragrans, Gentian Root, Caulis Spatholobi, Jujube fruit, Dongquai, Mongolian milkvetch, and Szechuan lovage.

### Fang Feng Thang Remedy

Ingredients: Fang Feng, Qiang Huo, Cinnamon, Dongquai, Ginger, Pueraria lobata, Gentian Root, Liquorice, Almond, Poria cocos wolf.

### Du Huo Sang Ji Sheng Thang Remedy

Ingredients: Du Huo, Fang Feng, Sang Ji Sheng, Xi Xin, Gentian Root, Dongquai, Dangshen, Tu Fu-ling, Ox Knee, Hardy rubber tree, Cinnamon, Chinese foxglove, Chinese peony, Liquorice, Radix Aconiti lateralis praeparata.

### Eight Ingredients Remedy

Ingredients: Clematis sinensis Osbeck, Bai Zhu, Fu-ling, Ox Knee, Cibotium barometz, Rubiaceae, Dioscorea opposita, Cornaceae, Cinnamon, Radix Aconiti lateralis praeparata.

### Tam Ty T Remedy Hang

Ingredients: Dipsacus japonicus, Fang Feng, Xi Xin, Poria cocos wolf, Chinese peony, Ox Knee, Gentian Root, Szechuan lovage, Ginger, Hardy rubber tree, Cinnamon, Dangshen, Dongquai, Mongolian milkvetch, Liquorice, Chinese foxglove, Du Huo.

### Cinnamon Rhizoma Anemarrhena Thang Remedy

Ingredients: Cinnamon, Chinese peony, Liquorice, Bai Zhu, Fang Feng, Ginger, Herba Ephedrae, Rhizoma Anemarrhena, Radix Aconiti lateralis praeparata.

### Dongquai Niem Thong Thang Remedy

Ingredients: Bai Zhu, Pueraria lobata, Glycyrrhiza uralensis Fisch, Dang Shen Giseng, Dongquai, Scutellaria baicalensis Georg, Sophora flavescens Ait, Qiang Huo, Adenosma caeruleum R. Br., Fang Feng, Atractylodes lancea, Alisma plantago-aquatica, Rhizoma Anemarrhena, Basidiomycetes, ect.

Use of traditional medicine remedies in the treatment of osteoarthritis pain is a method with good treatment results. However, for the traditional remedies, it takes a lot of time to boil raw ingredients for obtaining medicine drink, so it is inconvenient for patients. Therefore, there is always a need to improve and create products for the treatment of osteoarthritis that are more effective in treating these diseases and easy to use by the patients.

### SUMMARY OF THE INVENTION

The purpose of the present invention s to overcome the disadvantages mentioned above.

For this purpose, according to a first aspect of the present invention, the present invention provides a medicine composition for treatment of bone and joint diseases (i.e. osteoarthritis) originated from medicinal that has its main ingredients as dried extracts of the medicinal herbs, tortoiseshell extract, mixed bone extract, deer antler extract, wherein the dried extract of the medicinal herbs plays a role of active ingredients with therapeutic effects, while the extracts of animal bones has an adding effect to strengthen bones and improve health. The medicinal herbs extracts are obtained from herbs that have beneficial effects on bones and joints, in particular:

Huang Bai (bark), known also as Cortex Phellodendri—has the effects of heat-clearing, detoxifying, sedating, treating tuberculosis, cholera, anti-inflammatory, treating cholestatic jaundice, hemorrhoids, back pain, leg weakness, diarrhea, dysentery, spermatorrhea in men, urinary retention, blood in urine, boils, sores on tongue, sores on mouth, red swollen eyes, dull pain in bones, night sweats.

Liquorice (root/rhizome), known also as Radix Glycyrrhizae—has the effects of strengthening spleen and stomach when used with Dangshen, Poria cocos wolf, Bai Zhu; nourishing blood, used in treating mental illness, weakness palpitations, sadness—in combination—with Dangshen, E Jiao, Ophiopogon japonicus, Cinnamon; Heat-clearing, detoxification: used in treating boils, swelling pain; treating stomach pain, gastrointestinal ulcer, abdominal pain, combined with Chinese peony for treating tendon contracture; Adjusting taste, reducing side effects.

Hardy rubber tree (bark), known also as Cortex Eucommiae Glycyrrhizae—has the effects of Tonic for kidney and liver: treating spermatorrhea, spermatorrhea, impotence due to kidney failure; Strengthening tendons and bones, treating back pain, knee pain caused by kidney failure when combining them with Sang Ji Sheng, Ox Knee; treating easy miscarriage, premature birth, commonly used with E Jiao, Sang Ji Sheng for treating pregnancy bleeding; Treating hypertension, cerebral infarction, senile.

Ox Knee (root), known also as Radix Achyranthis bidentatae—has the effects of Activating blood circulation: regulating menstruation, treating cessation of menstruation, dysmenorrhea; Strengthening tendons and bones: treating joint pains, especially for leg joints; Detoxification, anti-inflammatory: treating sore throat swelling pain, sores on mouth, sore gums; Lowering blood pressure: treating hypertension due to its ability to lower cholesterol; Diuretic: treating diseases of blood in urine, calculus, painful urination.

Fang Feng (root), known also as Radix Saposhnikoviae divaricatae—has the effects of treating colds, headaches, body aches when used with marjoram; Treating neuralgia, muscle spasticity, pain in joints; Allergy treatment, cold sore rash when used with Cinnamon; treating convulsions, tetanus; Detoxification: Arsenic, reducing toxicity.

Poria cocos wolf (fruit body), known also as Poria cocos—has the effects of Osmotic diuresis: treating infections in kidneys, bladder, blood in the urine, frequent urination, cloudy urine, little urine, red, cloudy urine when used in combination with Cinnamon, Bai Zhu; Strengthening spleen and stomach pain causing diarrhea; Sedating: treating sleep problems, insomnia when used in combination with Dongquai, longan, ect. For patients with bone and joint degeneration and pains they often have problems with kidney failure, Poria cocos wolf helps sedation, reducing pains and stress.

Chinese foxglove (rhizome), known also as Rhizoma Rehmanniae—has the effects of heat-clearing in blood; Treating high fever for a long time leading to dehydration; Treating long-term cough, autonomic nervous disorder due to tuberculosis; Treating bleeding due to infectious fever: nosebleeds, bloody dysentery, hemoptysis; Treating constipation due to visceral heat, or high fever causing dehydration constipation; Detoxification of the body, treating sore throat, pimples; Safe pregnancy when infected with toxic bacteria; Cardiomyopathy mainly affects the myocardium; Diuretic due to the above-mentioned cardiomyopathy and vascular dilating effects in kidneys; Hypoglycemia; Inhibiting cutaneous fungal infection.

Sang Ji Sheng (whole tree), known also as Herba Loranthi Gracifilolii—has the effects of treating joint and bone pains, peripheral neuralgia; back pain in the elderly; children with delayed walk, delayed teeth grow; Nourishing blood for safe pregnancy, used when bleeding during pregnancy, prevention of miscarriage or premature birth; Lowering blood pressure used in the case of hypertension when combining them with Ox Knee.

Gentian Root (root), known also as Radix Gentianae macrophyllae—has the effects of treating joint pains, neuralgia when used with Sang Ji Sheng, Szechuan lovage; Treating dull pain in bones, fever in the afternoons due to internal heat generation—used together with Rhizoma Anemarrhena, Foxglove; Treating liver infections: viral hepatitis, biliary tract inflammation.

Peach blossom (blossom), known also as Flos Prunus persicae—has the effects of diuretics, helminths, breaking kidney stones, clearing blood flow, treating madness, treating back pain in the waist. In traditional medicine, to treat diseases related to bones and joints, it is often necessary to strengthen the function of the kidney organs.

Dried tangerine peel (peel), known also as Pericarpium citri reticulatae—has the effects of supplementing the spleen and stomach, regulating the digestive tract; It is a medicine that helps to guide the drug in the remedy.

Chinese peony (root), known also as Radix Pacomiae Lactiflorae—has the effects of blood tonic: treating people with anemia then in combination with Foxglove, Dongquai, Radix Polygoni Multiflori, Szechuan lovage; Regulating menstruation, treating menstrual irregularities, dysmenorrhea; Stop bleeding: treating bleeding disorder, hemoptysis, bloody stools, blood in the urine, menorrhagia, bloody hemorrhoids; pain relief: treating pains like: stomach pain, rib pain; abdominal pain diarrhea due to neural problems, limbs convulsions then in combination with Liquorice; Treating night sweat; Liver tonic: treating headaches, insomnia.

Dangshen (root), known also as Radix Codonopsis pilosulae—has the effects of lung weakness causing cough, asthma for a long time caused by kidney failure; Treating spleen weakness, poor appetite, loose stools, fatigue, thirst, long-term illness, body weakness; Complementary nourishing blood treatment for rectal prolapse, uterine prolapse; Nourishing the body, diuretic, treating cough, eliminating phlegm.

Du Huo (root), known also as Radix Angelicae Tuhuo—has the effects of treating joint pains, neuralgia or pains from back down (kidney failure); Treating cold causing headaches, fever, back pain.

Dongquai (root), known also as Radix Angelicae sinensis—has the effects of blood tonic.

Cinnamon (branch), known also as Ramulus cinnamoni—has the effects of treating colds; Treating joint pains, nerve pains, muscle spasticity due to cold; Treating cough and stimulating expectoration; diuretic; enhancing gasification in the bladder in combination with Bai Zhu.

Xi Xin (root, rhizome) known also as Radix et Rhizoma Asari—has the effects of treating colds and flu causing body aches, headaches, nasal congestion; Treating cough and thin phlegm, bronchial asthma; Treating joint pains and neuralgia due to cold.

Szechuan lovage (rhizome), known also as Rhizoma ligustici Wallichi—has the effects of blood tonic; Treating headaches, body aches, joint pains due to rheumatism; treatmenting rib pain; Anti-inflammatory treatment of pimples; Blood tonic.

Dwarf umbrella tree (bark), known also as Cortex Scheflerae heptaphyllae—has the effects of strengthening tendons and bones; treating back pain, bone pain due to rheumatism, tendon contracture, swelling pain, or swelling pain due to trauma.

The above-mentioned medicinal herbs are treated into extracts, then combined with tortoiseshell extract, mixed extracts cattle bones such as buffalo, cow, goat, pig, deer antler extract and excipients to obtain the medicine composition for treatment of bone and joint diseases. By taking numerous production trials, the inventor has found a production process with qualitative and quantitative ingredients and optimal conditions to yield the final medicine composition of the present invention that is highly effective in the treatment of musculoskeletal diseases. Subsequent preclinical and clinical trials have demonstrated superior efficacy of the medicine composition of the present invention over known medicinal products and preparations.

More specifically, according to a first aspect of the present invention, the present invention provides a medicine composition for treatment of bone and joint diseases originated from medicinal materials as said in Claim 1, that consists of (% by weight):
dried extract of the medicinal herbs: 40-75, more preferably 45-70, most preferably 50-65;
tortoiseshell extract: 0.1-20, more preferably 2-18, most preferably 3-15;
mixed bone extract: 0.1-20, more preferably 2-18, most preferably 3-15;
deer antler extract: 0.1-20, more preferably 2-18, most preferably 3-15;
salix alba extract: 5-25, more preferably 7-22, most preferably 10-20;
nattokinaza: 0.1-10, more preferably 0.5-8, most preferably 0.7-7;
Na-benzoate: 0-0.5, more preferably 0.05-0.4, most preferably 0.1-0.3,
   wherein the herbal medicine mixture comprises the ingredients of: Huang Bai (bark), Liquorice (root/rhizome), Hardy rubber tree (bark), Ox Knee (root), Fang Feng (root), Poria cocos wolf (fruit body), Chinese foxglove (rhizome), Sang Ji Sheng (whole tree), Gentian Root (root), Rhizoma Anemarrhena (rhizome), Peach blossom (blossom), Dried tangerine peel (peel), Chinese peony (root), Dangshen (root), Ginseng (root) Du Huo (root), Dongquai (root), Cinnamon (bough), Xi Xin (root, rhizome) and Szechuan lovage (rhizome), Dwarf umbrella tree (root).

According to a preferable embodiment, as said in Claim 2, said herbal medicine mixture has an ingredient ratio as follows (% by weight):

Huang Bai: 10-45; Liquorice: 0.1-10; Hardy rubber tree: 0.5-15; Ox Knee: 0.1-10; Fang Feng: 0.1-10; Poria cocos wolf: 0.5-15; Chinese foxglove: 0.5-15; Sang Ji Sheng: 0.1-10; Gentian Root: 0.1-10; Rhizoma Anemarrhena: 0.1-10; Peach blossom: 0.5-15; Dried tangerine peel: 0.5-15; Chinese peony: 1-25; Dangshen: 1-15; Ginseng: 1-15; Du Huo: 0.1-10; Dongquai: 1-15; Cinnamon: 0.1-10; Xi Xin: 0.1-5; Szechuan lovage: 0.1-10, and Dwarf umbrella tree: 0.1-10.

According to a preferable embodiment, as said in Claim 3, more preferably the herbal medicine mixture has an ingredient ratio as follows (% by weight):

Huang Bai: 15-40; Liquorice: 0.5-8; Hardy rubber tree: 0.7-12; Ox Knee: 0.5-8; Fang Feng: 0.5-8; Poria cocos wolf: 0.7-12; Chinese foxglove: 0.7-12; Sang Ji Sheng: 0.5-8; Gentian Root: 0.5-8; Rhizoma Anemarrhena: 0.5-8; Peach blossom: 0.7-12; Dried tangerine peel: 0.7-12; Chinese peony: 5-20; Dangshen: 1.5-12; Ginseng: 1.5-12; Du Huo: 0.5-8; Dongquai: 1.5-12; Cinnamon: 0.5-8; Xi Xin: 0.2-4; Szechuan lovage: 0.5-8, and Dwarf umbrella tree: 0.5-8.

According to a preferable embodiment, as said in Claim 4, most preferably the herbal medicine mixture has an ingredient ratio as follows (% by weight):

Huang Bai: 20-35; Liquorice: 0.7-5; Hardy rubber tree: 1-10; Ox Knee: 0.7-5; Fang Feng: 0.7-5; Poria cocos wolf 1-10; Chinese foxglove: 1-10; Sang Ji Sheng: 0.7-5; Gentian Root: 0.7-7; Rhizoma Anemarrhena: 0.7-7; Peach blossom: 1-10; Dried tangerine peel: 1-10; Chinese peony: 8-18; Dangshen: 2-10; Ginseng: 2-10; Du Huo: 0.7-7; Dongquai: 2-10; Cinnamon: 0.7-5; Xi Xin: 0.3-3; Szechuan lovage: 0.7-7; and Dwarf umbrella tree: 0.7-7.

According to a second aspect of the present invention, the present invention provides a process for producing medicine composition for treatment of bone and joint diseases originated from medicinal materials as said in Claim 5, said process comprise the following steps:

i) Preparing medicinal herbs of: Huang Bai (bark), Liquorice (root/rhizome), Hardy rubber tree (bark), Ox Knee (root), Fang Feng (root), Poria cocos wolf (fruit body), Chinese foxglove (rhizome), Sang Ji Sheng (whole tree), Gentian Root (root), Rhizoma Anemarrhena (rhizome), Peach blossom (blossom), Dried tangerine peel (peel), Chinese peony (root), Dangshen (root), Ginseng (root), Du Huo (root), Dongquai (root), Cinnamon (bough), Xi Xin (root, rhizome), Szechuan lovage (rhizome), and Dwarf umbrella tree (root) mixed in a predefined proportion, washed, dried, then stirred in a hot pan;

ii) Extracting the herbal medicine mixture to obtain a dried extract of the herbal medicine mixture;

iii) Preparating mixed bone extract, tortoiseshell extract and deer antler extract:
washing animal bones, tortoiseshells, deer antlers from trash and tissues, then incubating them with 10% ethanol 45% for 2 hours, then feeding them into a boiling pot, periodically extracting the boiled extract every 24 hours for 3 times, during said process boiling water is added to keep the boiled material under the water, to obtain mixed extracts;
evaporating the mixed extracts to a moisture content of 20-25%, then adding 0.5% preservatives nipagin/nipasol at a ratio of 9/1 or sodium benzoate dissolved in ethanol 70%, stirring the mixture to evenly distributed state, then evaporating evenly distributed mixture to a moisture content of about 25%;
if the batches of extracts are took place continuously, the third extraction can be used as a solvent for the first extraction of the following batch; and

iv) Obtaining the medicine compositions for treatment of bone and joint diseases by mixing the semi-products obtained from the above said steps at the following ration (% by weight):
dried extract of the medicinal herbs: 40-75, more preferably 45-70, most preferably 50-65;
tortoiseshell extract: 0.1-20, more preferably 2-18, most preferably 3-15;
mixed bone extract: 0.1-20, more preferably 2-18, most preferably 3-15;
deer antler extract: 0.1-20, more preferably 2-18, most preferably 3-15;
salix alba extract: 5-25, more preferably 7-22, most preferably 10-20;
nattokinaza: 0.1-10, more preferably 0.5-8, most preferably 0.7-7;
Na-benzoate: 0-0.5, more preferably 0.05-0.4, most preferably 0.1-0.3.

According to a preferable embodiment, as mentioned in Claim 6, for producing solid extracts, step preparating the medicinal herbs consists of grinding mixture of the medicinal herbs into a powder, then sieving through a sieve to separate them into particles with a particle size of <0.2 mm (fine extract powder) and those with a particle size ≥0.2 mm (crude powder).

According to a preferable embodiment, as mentioned in Claim 7, for producing solid extracts, step extracting mixture the medicine composition consists of preparating binders, this step is carried out as follows:
preparating extract mixture of the medicinal herbs by washing the medicinal herbs and then chopping them, then in combination with the crude powder having a particle size of ≥0.2 mm which has been feeded into damp cloth bags and sealed;
putting the bags of the crude powder and the chopped medicinal herbs into an extraction pot, adding water to about 20 cm above the surface of the medicinal herbs, heating up to boiling point and keeping boiling for about 3 hours, then collecting an extract;
repeating the extraction process 3 times, combining the extracts and filtering them through a sieve of size 0.2 mm, concentrating semi-solid extracts by direct evaporation to about 30% moisture content;
dissolving the bone extract obtained in step (iii) into boiling water, adding said semi-solid extracts mixture of the medicinal herbs with 30% moisture content and heating up until evenly dispersed into a homogeneous mixture (Mixture A) to be used for making the binders;
preparating NaCMC binder by adding nipagin, nipasol into the water purified water, then stirring until completely dissolved state, then slowly adding NaCMC while stirring the solution to evenly distributed state, letting the solution swell for about 8-12 hours to obtain a NaCMC binder;
preparating starch binder by mixing purified water, amidon, sodium benzoate, cassava starch in an appropriate proportion, well stirring the mixture while heating it up to form the starch binder;
mixing the binders by thoroughly mixing Completely swelled NaCMC binder with the starch binder and extract Mixture A when they are still hot while well stirring to obtain the binder mixture that is used for producing the medicine composition.

According to a preferable embodiment, as mentioned in Claim 8, for producing hard capsules, step extracting the herbal medicine mixture to obtain the medicinal herbs extracts is carried out by two stages as follows:
by supercritical CO2 extraction:
washing the medicinal herbs, feeding them into extraction pot for supercritical CO2 extraction in 2 repeated cycles, each cycle is extracted with co-solvent EtOH/CO₂ 0-5% (weight) under the following conditions: pressure 20-40 MPas, temperature 40-70° C., duration 60-180 minutes, flow CO₂ 100-500 kg/h; after that collecting the extracts of the 2 cycles then packing them into 2-layer PE bags or aluminum bags; the residues of said supercritical C02 extraction will be used for the following water extraction;
by water extraction:
pouring water over 10-15 cm above the surface of the residue and boiling them for 3 hours of boiling, then collecting the first extract and adding water to boil for the second time, repeating the extraction two times; combining the extracts, filtering through a coarse cloth and concentrating the extracts until obtaining the extracts with a moisture content of about 20%; adding nipagin and nipasol or sodium benzoate dissolved in ethanol 96% v\acute{ι}ra dů, stirring well; casting into extract cakes to obtain concentrated extract a moisture content of 20%;
combining the concentrated extract of 20% moisture content the extract of supercritical CO2 extraction to form a mixture of the concentrated medicinal herbs that is dried by a static oven or spray-dried, at a high temperature of about 85° C. to dry, letting it cool and then grind it finely with a hammer mill and sieve it through a 0.2 mm sieve to obtain a mixture of powdered extract of the medicinal herbs used to prepare preparations in the following stage.

According to a preferable embodiment, as mentioned in Claim 9, the herbal medicine mixture has the following proportions of ingredients (% by weight):

Huang Bai: 10-45; Liquorice: 0.1-10; Hardy rubber tree: 0.5-15; Ox Knee: 0.1-10; Fang Feng: 0.1-10; Poria cocos wolf: 0.5-15; Chinese foxglove: 0.5-15; Sang Ji Sheng: 0.1-10; Gentian Root: 0.1-10; Rhizoma Anemarrhena: 0.1-10; Peach blossom: 0.5-15; Dried tangerine peel: 0.5-15; Chinese peony: 1-25; Dangshen: 1-15; Ginseng: 1-15, Du Huo: 0.1-10; Dongquai: 1-15; Cinnamon: 0.1-10; Xi Xin: 0.1-5; Szechuan lovage: 0.1-10, and Dwarf umbrella tree: 0.1-10.

According to a preferable embodiment, as mentioned in Claim 10, more preferably the herbal medicine mixture has the following proportions of ingredients (% by weight):

Huang Bai: 15-40; Liquorice: 0.5-8; Hardy rubber tree: 0.7-12; Ox Knee: 0.5-8; Fang Feng: 0.5-8; Poria cocos wolf: 0.7-12; Chinese foxglove: 0.7-12; Sang Ji Sheng: 0.5-8; Gentian Root: 0.5-8; Rhizoma Anemarrhena: 0.5-8; Peach blossom: 0.7-12; Dried tangerine peel: 0.7-12; Chinese peony: 5-20; Dangshen: 1.5-12; Ginseng: 1.5-12; Du Huo: 0.5-8; Dongquai: 1.5-12; Cinnamon: 0.5-8; Xi Xin: 0.2-4; Szechuan lovage: 0.5-8; and Dwarf umbrella tree: 0.5-8.

According to a preferable embodiment, as mentioned in Claim 11, most preferably the herbal medicine mixture has the following proportions of ingredients (% by weight):

Huang Bai: 20-35; Liquorice: 0.7-5; Hardy rubber tree: 1-10; Ox Knee: 0.7-5; Fang Feng: 0.7-5; Poria cocos wolf: 1-10; Chinese foxglove: 1-10; Sang Ji Sheng: 0.7-5; Gentian Root: 0.7-7; Rhizoma Anemarrhena: 0.7-7; Peach blossom: 1-10; Dried tangerine peel: 1-10; Chinese peony: 8-18; Dangshen: 2-10; Ginseng: 2-10; Du Huo: 0.7-7; Dongquai: 2-10; Cinnamon: 0.7-5; Xi Xin: 0.3-3; Szechuan lovage: 0.7-7; and Dwarf umbrella tree: 0.7-7.

According to a preferable embodiment, as mentioned in Claim 12, to prepare the medicine composition in the form of solid extracts, the preparation step is carried out as follows:
dry mixing fine extract powder in a mixer for about 10 to 35 minutes for obtaining a homogeneous dry mixture of extract powders;
slowly adding all binders to the obtained dry mixture of extract powders, mixing for about 5 to 15 minutes for obtaining a homogeneous wet mass of the extracts;
dividing the obtained wet mass of the extracts by a pelletizing machine; sieve to classify the obtained extract pellets to obtain extract pellets of size 4-6.0 mm;
drying the obtained extract pellets obtained at a temperature of 65-90° C. to a moisture content of not over 10% to obtain dry extract pellets;
then sorting the obtained dry extract pellets by a pellet sorting machine, collecting the extract pellets with the required size.

According to a preferable embodiment, as mentioned in Claim 13, to prepare the medicine composition in the form of hard capsules, the preparation step is carried out as follows:
thoroughly mixing fermented soybean (nattokinase), dry powder of the medicinal herbs and powders of bone extract and canxi carbonat according to the principle of geometric dilution;
thoroughly mixing cassava starch with the obtained mixture according to the principle of geometric dilution, finally with a mixture of glidants consisting of talc powder or erosil and magnesi stearat (already mixed) to obtain a powdered mixture for encapsulation;
encapsulating the powdered mixture by an automatic encapsulation machine to obtain capsules with the encapsulation powder weight of about 0.786 g→0.913 g (0.85 g±7.5%).

### DETAILED DESCRIPTION OF THE INVENTION

The present invention now will be described in more detail through the preferred embodiments which should not be considered as of the limitations of the invention.

Accordingly, the process for producing medicine composition for treatment of bone and joint diseases originated from medicinal materials including the following steps:

i) Preparing medicinal herbs of: Huang Bai (bark), Liquorice (root/rhizome), Hardy rubber tree (bark), Ox Knee (root), Fang Feng (root), Poria cocos wolf (fruit body), Chinese foxglove (rhizome), Sang Ji Sheng (whole tree), Gentian Root (root), Rhizoma Anemarrhena (rhizome), Peach blossom (blossom), Dried tangerine peel (peel), Chinese peony (root), Dangshen (root), Ginseng (root) Du Huo (root), Dongquai (root), Cinnamon (bough), Xi Xin (root, rhizome), Szechuan lovage (rhizome) and Dwarf umbrella tree (rhizome) mixed in a predefined proportion, washed, dried, then stirred in a hot pan.

According to a preferable embodiment, the herbal medicine mixture above has the following proportions of ingredients (% by weight): Huang Bai: 10-45; Liquorice: 0.1-10; Hardy rubber tree: 0.5-15; Ox Knee: 0.1-10; Fang Feng: 0.1-10; Poria cocos wolf: 0.5-15; Chinese foxglove: 0.5-15; Sang Ji Sheng: 0.1-10; Gentian Root: 0.1-10; Rhizoma Anemarrhena: 0.1-10; Peach blossom: 0.5-15; Dried tangerine peel: 0.5-15; Chinese peony: 1-25; Dangshen: 1-15; Ginseng: 1-15 Du Huo: 0.1-10; Dongquai: 1-15; Cinnamon: 0.1-10; Xi Xin: 0.1-5; Szechuan lovage: 0.1-10 and Dwarf umbrella tree: 0.1-10.

According to a preferable embodiment, more preferably the herbal medicine mixture has the following proportions of ingredients (% by weight): Huang Bai: 15-40; Liquorice: 0.5-8; Hardy rubber tree: 0.7-12; Ox Knee: 0.5-8; Fang Feng: 0.5-8; Poria cocos wolf: 0.7-12; Chinese foxglove: 0.7-12; Sang Ji Sheng: 0.5-8; Gentian Root: 0.5-8; Rhizoma Anemarrhena: 0.5-8; Peach blossom: 0.7-12; Dried tangerine peel: 0.7-12; Chinese peony: 5-20; Dangshen: 1.5-12; Ginseng: 1.5-12; Du Huo: 0.5-8; Dongquai: 1.5-12; Cinnamon: 0.5-8; Xi Xin: 0.2-4; Szechuan lovage: 0.5-8 and Dwarf umbrella tree 0.5-8.

According to a preferable embodiment, most preferably the herbal medicine mixture has the following proportions of ingredients (% by weight): Huang Bai: 20-35; Liquorice: 0.7-5; Hardy rubber tree: 1-10; Ox Knee: 0.7-5; Fang Feng: 0.7-5; Poria cocos wolf: 1-10; Chinese foxglove: 1-10; Sang Ji Sheng: 0.7-5; Gentian Root: 0.7-7; Rhizoma Anemarrhena: 0.7-7; Peach blossom: 1-10; Dried tangerine peel: 1-10; Chinese peony: 8-18; Dangshen: 2-10; Ginseng: 2-10; Du Huo: 0.7-7; Dongquai: 2-10; Cinnamon: 0.7-5; Xi Xin: 0.3-3; Szechuan lovage: 0.7-7 and Dwarf umbrella tree 0.7-7.

ii) Extracting the herbal medicine mixture to obtain a dried extract of the herbal medicine mixture.

iii) Preparating mixed bone extract, tortoiseshell extract and deer antler extract:
washing animal bones, tortoiseshells, deer antlers from trash and tissues, then incubating them with 10% ethanol 45% for 2 hours, then feeding them into a boiling pot, periodically extracting the boiled extract every 24 hours for 3 times, during said process boiling water is added to keep the boiled material under the water, to obtain mixed extracts;
evaporating the mixed extracts to a moisture content of 20-25%, then adding 0.5% preservatives nipagin/nipasol at a ratio of 9/1 dissolved in ethanol 70%, stirring the mixture to evenly distributed state, then evaporating evenly distributed mixture to a moisture content of about 25%;
if the batches of extracts are took place continuously, the third extraction can be used as a solvent for the first extraction of the following batch; and

iv) Obtaining the medicine compositions for treatment of bone and joint diseases by mixing the semi-products obtained from the above said steps at the following ration (% by weight):
dried extract of the medicinal herbs: 40-75, more preferably 45-70, most preferably 50-65;
tortoiseshell extract: 0.1-20, more preferably 2-18, most preferably 3-15;
mixed bone extract: 0.1-20, more preferably 2-18, most preferably 3-15;
deer antler extract: 0.1-20, more preferably 2-18, most preferably 3-15;
salix alba extract: 5-25, more preferably 7-22, most preferably 10-20;
nattokinaza: 0.1-10, more preferably 0.5-8, most preferably 0.7-7;
Na-benzoate: 0-0.5, more preferably 0.05-0.4, most preferably 0.1-0.3.

The above said process is the general process for producing preparations for treatment of bone and joint diseases. However, the present invention does not only provide said general process but also provides certain embodiments for producing the medicine composition in two preferred forms, namely solid extracts and hard capsules.

Accordingly, for producing solid extracts, according to a preferable embodiment, in the step of preparating the medicinal herbs mixture is ground into a powder, then sieved through a sieve to separate them into particles with a particle size of <0.2 mm (fine extract powder) and those with a particle size ≥0.2 mm (crude extract powder). Sieves used can be any type with a sieve mesh size of 0.2 mm, preferably stainless steel sieves to ensure food hygiene and safety.

Wherein, in the step of extracting mixture the medicine composition for producing solid extracts, including preparating binders, this step is carried out as follows:
preparating extract mixture of the medicinal herbs by washing the medicinal herbs and then chopping them, then in combination with the crude powder having a particle size of ≥0.2 mm as obtained above, then feeding into damp cloth bags and sealed;
putting the bags of the crude powder and the chopped medicinal herbs into an extraction pot, adding water to about 20 cm above the surface of the medicinal herbs, heating up to boiling point and keeping boiling for about 3 hours, then collecting an extract;
repeating the extraction process 3 times, combining the extracts then sorting through a sieve with a size 0.2 mm, concentrating semi-solid extracts by direct evaporation to about 30% moisture content;
dissolving the bone extract obtained in step (iii) into boiling water, adding said semi-solid extracts mixture of the medicinal herbs with 30% moisture content and heating up until evenly dispersed into a homogeneous mixture (Mixture A) to be used for making the binders;
preparating NaCMC binder by weighing the purified water, adding nipagin, nipasol into the water then stirring until completely dissolved state, slowly adding NaCMC while stirring the solution to evenly distributed state, letting the solution swell for about 8-12 hours to obtain a NaCMC binder;
preparating starch binder by mixing purified water, amidon, sodium benzoate, cassava starch in an appropriate proportion, well stirring the mixture while heating it up to form the starch binder;
mixing the binders by thoroughly mixing Completely swelled NaCMC binder with the starch binder and extract Mixture A when they are still hot while well stirring to obtain the binder mixture that is used for producing the medicine composition.

Wherein, to prepare the medicine composition in the form of solid extracts, the preparation step is carried out as follows:
Weighing prepared fine extract powders then loading them into a mixer, mixing the powders for about 10 to 35 minutes for obtaining a homogeneous dry mixture of extract powders;
Slowly adding all binders to the obtained dry mixture of extract powders, mixing for about 5 to 15 minutes for obtaining a homogeneous wet mass of the extracts;
Dividing the obtained wet mass of the extracts by a pelletizing machine;
Sieve to classify the obtained extract pellets to obtain extract pellets of size 4-6.0 mm;
Drying the obtained extract pellets obtained at a temperature of 65-90° C. to a moisture content of not over 10% to obtain dry extract pellets;
Then sorting the obtained dry extract pellets by a pellet sorting machine, collecting the extract pellets with the required size.

The medicine composition obtained by above said process is the medicine composition of the present invention in the form of solid extracts.

The obtained solid extracts can ben further processed to have eye-catching colors and appearance to meet the consumer tastes, such as color coating and capsule coating.

For color coating, the following steps can be carried out:
Mixing ethanol 50° according to the formulation, adding activated carbon, then stirring well continuously to ensure that the suspension does not settle, and is always homogeneous during the coating process. The resulting colored suspension B is used for color coating.
Putting the obtained pellets into a cleaned pot.
Letting the pellets run while spraying the color suspension into the running pellets (the color is continuously stirred in the stirrer during the spraying process to prevent sedimentation) to reach a uniformity of the color on the surface of the pellets until the color liquid is gone. Checking color uniformity.
Keeping the colored pellets running in the pot until the alcohol is completely evaporated.

For polishing the colored pellets, the following steps can be carried out:
Heating the color coated pellets up to about 50-80° C. for about 3-5 hours;
Applying melted solid paraffin evenly over the pellet surface, turning off the heat supply;
Keeping the pot with said pellets inside spined until the pellets are evenly polished and completely cooled.
Transferring the polished pellets into 2-layer PE bags then sealing the bags;
Taking samples for testing of semi-finished products, if satisfactory, moving to the packaging and storing.

For the embodiment of preparing the composition in the form of hard capsules, in the step of extracting the herbal medicine mixture to obtain the medicinal herbs extracts to make hard capsules, the extraction procedure can be proceed in the following two stages:

### Supercritical CO2 Extraction:

Washing the medicinal herbs, feeding them into extraction pot for supercritical C02 extraction in 2 repeated cycles, each cycle is extracted with co-solvent EtOH/CO₂ 0-5% (weight) under the following conditions: pressure 20-40 MPas, temperature 40-70° C., duration 60-180 minutes, flow CO₂ 100-500 kg/h; then collecting the extracts of the 2 cycles, filtering and concentrating the extracts into concentrated extract with a moisture content of about 20%. The residues of said supercritical C02 extraction will be used for the following water extraction.

### Water Extraction:

Pouring water over 10-15 cm above the surface of the residue and boiling them for 3 hours of boiling, then collecting the first extract and adding water to boil for the second time, repeating the extraction two times; combining the extracts, filtering through a coarse cloth and concentrating the extracts until obtaining the extracts with a moisture content of about 20%.

Adding in to the concentrated extract obtained above nipagin and nipasol or sodium benzoate dissolved in enough ethanol 96%, stirring well; casting into extract cakes to obtain concentrated extract a moisture content of 20%.

Mixing the concentrated extract obtained above with a moisture content of 20% with the extract of supercritical C02 extraction to form a mixture of the concentrated medicinal herbs that is dried by a static oven or spray-dried, at a high temperature of about 85° C. to dry, letting it cool and then grind it finely with a hammer mill and sieve it through a 0.2 mm sieve to obtain a mixture of powdered extract of the medicinal herbs used to prepare preparations in the following stage.

To obtain the composition in a form of hard capsules, the following preparation steps are carried out:
Fermented soybean paste (nattokinase), dry powder of the medicinal herbs, powders of bone extract and canxi carbonat thoroughly mixing in an appropriate proportion according to the principle of geometric dilution;
Thoroughly mixing cassava starch with the obtained mixture according to the principle of geometric dilution, finally with a mixture of glidants consisting of talc powder or erosil and magnesi stearat (already mixed) to obtain a powdered mixture for encapsulation; and
Encapsulating the powdered mixture by an automatic encapsulation machine to obtain capsules with the encapsulation powder weight of about 0.786 g to 0.913 g (0.85 g±7.5%).

Another object of the present invention is a medicine composition for treatment of bone and joint diseases originated from medicinal materials that is produced by the process mentioned above, said medicine composition has its ingredients consisting of (% by weight):
dried extract of the medicinal herbs: 40-75, more preferably 45-70, most preferably 50-65;
tortoiseshell extract: 0.1-20, more preferably 2-18, most preferably 3-15;
mixed bone extract: 0.1-20, more preferably 2-18, most preferably 3-15;
deer antler extract: 0.1-20, more preferably 2-18, most preferably 3-15;
salix alba extract: 5-25, more preferably 7-22, most preferably 10-20;
nattokinaza: 0.1-10, more preferably 0.5-8, most preferably 0.7-7;
Na-benzoate: 0-0.5, more preferably 0.05-0.4, most preferably 0.1-0.3.

The herbal medicine mixture comprises the ingredients of: Huang Bai (bark), Liquorice (root/rhizome), Hardy rubber tree (bark), Ox Knee (root), Fang Feng (root), Poria cocos wolf (fruit body), Chinese foxglove (rhizome), Sang Ji Sheng (whole tree), Gentian Root (root), Rhizoma Anemarrhena (rhizome), Peach blossom (blossom), Dried tangerine peel (peel), Chinese peony (root), Dangshen (root), Ginseng (root), Du Huo (root), Dongquai (root), Cinnamon (bough), Xi Xin (root, rhizome), Szechuan lovage (rhizome) and Dwarf umbrella tree (root).

The herbal medicine mixture has raw ingredients for extracting medicine extracts with the ration (% by weight) as follows: Huang Bai: 10-45; Liquorice: 0.1-10; Hardy rubber tree: 0.5-15; Ox Knee: 0.1-10; Fang Feng: 0.1-10; Poria cocos wolf: 0.5-15; Chinese foxglove: 0.5-15; Sang Ji Sheng: 0.1-10; Gentian Root: 0.1-10; Rhizoma Anemarrhena: 0.1-10; Peach blossom: 0.5-15; Dried tangerine peel: 0.5-15; Chinese peony: 1-25; Dangshen: 1-15; Ginseng: 1-15, Du Huo: 0.1-10; Dongquai: 1-15; Cinnamon: 0.1-10; Xi Xin: 0.1-5; Szechuan lovage: 0.1-10 and Dwarf umbrella tree 0.1-10.

According to a preferable embodiment, more preferably the herbal medicine mixture has raw ingredients for extracting medicine extracts with the ration (% by weight) as follows: Huang Bai: 15-40; Liquorice: 0.5-8; Hardy rubber tree: 0.7-12; Ox Knee: 0.5-8; Fang Feng: 0.5-8; Poria cocos wolf: 0.7-12; Chinese foxglove: 0.7-12; Sang Ji Sheng: 0.5-8; Gentian Root: 0.5-8; Rhizoma Anemarrhena: 0.5-8; Peach blossom: 0.7-12; Died tangerine peel: 0.7-12; Chinese peony. 5-20; Dangshen: 1.5-12; Ginseng: 1.5-12; Du Huo: 0.5-8; Dongquai: 1.5-12; Cinnamon: 0.5-8; Xi Xin: 0.2-4; Szechuan lovage: 0.5-8 and Dwarf umbrella tree 0.5-8.

According to a preferable embodiment, most preferably the herbal medicine mixture has raw ingredients for extracting medicine extracts with the ration (% by weight) as follows: Huang Bai: 20-35; Liquorice: 0.7-5; Hardy rubber tree: 1-10; Ox Knee: 0.7-5; Fang Feng: 0.7-5; Poria cocos wolf: 1-10; Chinese foxglove: 1-10; Sang Ji Sheng: 0.7-5; Gentian Root: 0.7-7; Rhizoma Anemarrhena: 0.7-7; Peach blossom: 1-10; Dried tangerine peel: 1-10; Chinese peony: 8-18; Dangshen: 2-10; Ginseng: 2-10; Du Huo: 0.7-7; Dongquai: 2-10; Cinnamon: 0.7-5; Xi Xin: 0.3-3; Szechuan lovage: 0.7-7 and Dwarf umbrella tree 0.7-7.

### EXAMPLES

### Production Example 1

Some preferred formula embodiments of the medicine composition ingredients are shown in the below table.

| | Ingredients/ | | Formula | Formula | Formula | Formula | Formula | Formula | Formula | Formula |
|---|---|---|---|---|---|---|---|---|---|---|
| No. | Part of use | Content | 1 | 2 | 3 | 4 | 5 | 6 | 7 | 8 |
| 1 | Huang Bai (bark) | 10-45 | 10 | 14 | 18 | 25 | 28 | 34 | 40 | 45 |
| 2 | Liquorice (root/ | 0.1-10 | 10 | 7 | 7 | 5 | 3 | 1 | 0.5 | 0.1 |
| | rhizome) | | | | | | | | | |
| 3 | Hardy rubber | 0.5-15 | 0.5 | 15 | 11 | 11 | 9 | 7 | 5 | 3 |
| | tree (bark) | | | | | | | | | |
| 4 | Ox Knee (root) | 0.1-10 | 1 | 0.1 | 10 | 8 | 6 | 5 | 3 | 2 |
| 5 | Fang Feng (root) | 0.1-10 | 2 | 1 | 0.1 | 9 | 8 | 7 | 5 | 3 |
| 6 | Poria cocos | 0.5-15 | 7 | 5 | 3 | 0.5 | 14 | 12 | 11 | 7 |
| | wolf (fruit body) | | | | | | | | | |
| 7 | Chinese | 0.5-15 | 8 | 6 | 5 | 3 | 0.5 | 15 | 9 | 8 |
| | foxglove (rhizome) | | | | | | | | | |
| 8 | Sang Ji Sheng | 0.1-10 | 7 | 5 | 3 | 2 | 1 | 0.1 | 10 | 8 |
| | (whole tree) | | | | | | | | | |
| 9 | Gentian Root | 0.1-10 | 8 | 7 | 5 | 3 | 2 | 1 | 0.1 | 7 |
| | (root) | | | | | | | | | |
| 10 | Rhizoma | 0.1-10 | 8 | 8 | 7 | 5 | 3 | 2 | 1 | 0.1 |
| | Anemarrhena | | | | | | | | | |
| | (stem, root) | | | | | | | | | |
| 11 | Peach blossom | 0.5-15 | 14 | 8 | 7 | 5 | 2 | 0.5 | 7 | 9.6 |
| 12 | Dried tangerine | 0.5-15 | 2 | 0.9 | 0.5 | 10 | 2 | 3 | 1 | 0.5 |
| | peel (peel) | | | | | | | | | |
| 13 | Chinese | 1-25 | 3 | 5 | 9 | 2 | 1.5 | 1 | 1 | 1 |
| | peony (root) | | | | | | | | | |
| 14 | Dangshen (root) | 1-15 | 1 | 3 | 4 | 1 | 3 | 1 | 2 | 1 |
| 15 | Ginseng (root) | 1-15 | 1 | 3 | 4 | 2 | 3 | 1 | 2 | 1 |
| 16 | Du Huo (root) | 0.1-10 | 3 | 2 | 0.4 | 2 | 1 | 0.4 | 0.5 | 0.3 |
| 17 | Dongquai (root) | 1-15 | 2.5 | 2 | 1 | 1.5 | 6 | 2 | 1 | 1 |
| 18 | Cinnamon (bough) | 0.1-10 | 2 | 1 | 2 | 2 | 1.5 | 1 | 0.4 | 0.5 |
| 19 | Xi Xin (root, | 0.1-5 | 1 | 5 | 3 | 2 | 1.5 | 1 | 0.3 | 0.5 |
| | rhizome) | | | | | | | | | |
| 20 | Szechuan | 0.1-10 | 5 | 1 | 2 | 0.5 | 2 | 2 | 0.1 | 0.6 |
| | lovage (rhizome) | | | | | | | | | |
| 21 | Dwarf umbrella | 0.1-10 | 4 | 1 | 2 | 0.5 | 2 | 3 | 0.1 | 0.8 |
| | tree (root) | | | | | | | | | |
| | Total | | 100 | 100 | 100 | 100 | 100 | 100 | 100 | 100 |

### Production Example 2

Some formula for production of the invention

| No. | Ingredients | Content | Formula 1 | Formula 2 | Formula 3 | Formula 4 | Formula 5 | Formula 6 | Formula 7 |
|---|---|---|---|---|---|---|---|---|---|
| 1 | Dried extract | 40-75 | 40 | 45 | 55 | 60 | 65 | 70 | 75 |
| | mixture of | | | | | | | | |
| | the medicinal | | | | | | | | |
| | herbs | | | | | | | | |
| 2 | Tortoiseshell | 0-20 | 10 | 0 | 10 | 15 | 9.5 | 7 | 6 |
| 3 | Mixed bone | 0-20 | 10 | 14 | 0 | 5 | 5 | 7 | 4 |
| | extract | | | | | | | | |
| 4 | Deer antler | 0-20 | 4.5 | 10 | 9.5 | 0 | 5 | 7 | 9.5 |
| | extract | | | | | | | | |
| 5 | Salix alba | 5-25 | 25 | 22 | 18 | 15 | 12 | 8 | 5 |
| | extract | | | | | | | | |
| 6 | Nattokinase | 0.1-10 | 10 | 9 | 7 | 5 | 3 | 1 | 0.1 |
| 7 | Na-benzoate | 0-0.5 | 0.5 | 0 | 0.5 | 0 | 0.5 | 0 | 0.4 |
| | Total | | 100 | 100 | 100 | 100 | 100 | 100 | 100 |

### Experimental Example 1: Preclinical Study on Mice with One of Said Formulae—Experimental Drug TD0015 was Carried Out at the Department of Pharmacology, Hanoi Medical University

### Acute Toxicity:

Experimental drug TD0015: there was no acute toxicity at the dose of 37.5 g/kg;
LD50 has not been determined in mice with oral experimental drugs;
Index TI>15.6.

Thus, according to the regulations of the World Health Organization, experimental drug TD0015 is a medicinal drug with world-acceptable safety.

### Semi-Chronic Toxicity:

In both groups of mice, one group took the experimental drug at a clinically equivalent dose of 1.2 g/kg/day and the other group took a dose three times higher than the clinical dose (3.6 g/kg/day), continuously for 90 days. The results show:

### Both Doses of the Experimental Drug

reduced the weight of the mice compared with the biological controls, but did not adversely affect the overall condition of the mice;
did not change the results of tests to evaluate hematopoietic function (red blood cell count, hemoglobin content, hematocrit, mean red blood cell volume, white blood cell count, white blood cell formula) compared with the control group;
Did not change the results of liver function tests (total bilirubin, albumin in the blood of the mice) compared with the control group;
Both reagent doses reduced total cholesterol levels compared with controls;
Did not damage liver cells (AST, ALT activity in white blood of the mice) compared with control group;
Did not change the test results of creatinine in the blood of white rats after 90 days of continuous administration of the experimental drug compared with the control group;
There was no morphological damage when observing the macroscopic organs of the white rat compared with the control group;
Microstructure of liver and kidney of white rat: there was no damage to liver and kidney of white rat after 90 days of taking the experimental drug compared with the control group.

### Abnormal Toxicity:

The experimental drug sample produced by Sao Thai Duong Joint Stock Company meets the requirements for abnormal toxicity test according to Vietnam Pharmacopoeia IV, Appendix 13.5 and basic standards of preparation.

### Study on Analgesic and Anti-Inflammatory Effects:

Studying results on acute anti-inflammatory effects on white mice of the preparation by 2 methods: causing mouse paw edema and causing mouse peritonitis showed that:

A dose of 1.2 g/kg (clinically equivalent dose) orally for 5 consecutive days has an acute anti-inflammatory effect in white rats in the rat paw edema model, shown at 24 h and has acute anti-inflammatory effects when studied in a rat peritonitis model;

A dose of 3.6 g/kg (3 times higher than the clinical dose) orally for 5 consecutive days has an acute anti-inflammatory effect in white rats in the rat paw edema model, which are clearly shown after 2 h, 4 h, 24 h and has acute anti-inflammatory effects when studied in a rat peritonitis model.

### Chronic Anti-Inflammatory Effect:

A dose of 2.4 g/kg/day (clinically equivalent dose) has the effects of chronic anti-inflammatory in a model of chronic granulomatous inflammation in mice through reducing granuloma weight and number of inflammatory cells in the cortex of the granulomatous tumor;

A dose of 7.2 g/kg/day (3 times higher than the clinical dose) has the effects of chronic anti-inflammatory in a model of chronic granulomatous inflammation in mice through reducing granuloma weight, reducing the number of inflammatory cells in mice granulomatous area. This effect is equivalent to the effect of methylprednisolone at a dose of 10 mg/kg/day.

### Anti-Degeneration Effect:

The sample was handed over by Sao Thai Duong Joint Stock Company to the Department of Pharmacology at Hanoi Medical University, asking to evaluate the effects of joint degeneration treatment on rats at a dose of 1.2 g/kg and 3.6 g./kg drunk continuously for 6 weeks, the study results are as follows:
A dose of 1.2 g/kg has the effect of treating knee osteoarthritis in rats as shown by:

The effect of reducing knee swelling, pain relief and improving knee activity on a model using a pain tolerance meter;

The effect of pain relief at the knee joints on a model using a pain tolerance meter according to the Randall Selitto method;

The effect of improving the cartilage structure shown on the histopathology of the knee joint structure.
A dose of 3.6 g/kg has the effect of treating degeneration of knee joint in mice as shown by:
The effect of reducing knee swelling, reducing pain and improving knee operation on a model using a pain tolerance meter;
The effect of pain relief at the knee joint on a model using a pain tolerance meter according to the Randall Selitto method;
The effect of reducing the indexes of interleukin-1β and TNF-α which are specific indicators in degeneration of joints;
The effect of improving the cartilage structure is shown on the histopathology of the knee joint structure;
These effects were stronger than the effects created by the 1.2 g/kg dose.

### Experimental Example 2: Baseline Clinical Study

Study location: at Hai Hau District General Hospital—Nam Dinh.
Study design: This is an open-label, non-controlled study, using the medicine composition/experimental drug of the invention.

Taking the drug for 3 consecutive months, according to the manufacturer's recommended dose

### Study Subjects:

Volunteer patients, both inpatients and outpatients treated at Hai Hau District General Hospital—Nam Dinh who have signs of bone and joint disease: spondylosis, spine spines, degenerative disc and osteoporosis.
Study size: 60 patients
Sampling:

Study subjects were selected according to the following criteria: Patients with diseases such as joint and bone degeneration; spine spines; degenerative disc and osteoporosis.

### Elimination Criteria:

Pregnant patients and patients with congenital osteoarthritis.

### Data Collection Method:

The patient's medical record is made according to a certain single form; patients are monitored and re-examined once a week, assessed the degree of improvement in treatment of disease symptoms and drug tolerance; patients get laboratory tests once a month to assess the change in subclinical symptoms; Patients self-monitor the level of disease improvement at home.

### Data Collection Method:

Symptoms are assessed according to the checklist; biochemical, hematological tests, ect; tests to be able to diagnose the progression of the above-mentioned osteoarthritis.

### Managing, Processing and Analyzing Data:

The data is managed and processed according to the method of medical statistics.

### Study Ethics:

The patients voluntarily participate in the program (with commitment); The patient information is kept confidential for study purposes.

### Study Results:

### Epidemiological Features:

### Age: (Table 1)

| Age | Number of Patients | % on total |
|---|---|---|
| Range of 15-44 | 6 | 10 |
| Range of 45-64 | 45 | 75 |
| Over 65 | 9 | 15 |

The majority of patients are 45-64 years old (75%), through which we also see that the highest rate of osteoarthritis is in middle-aged and elderly people.

### Sex:

Among the patients with osteoarthritis, women always have a higher rate of disease than men. There are 21 male patients (35%) and 39 female patients (65%).

### Hospitalizing Reasons:

Joint pain, morning joint stiffness: 51/60 (85%); Joint creaking when moving: 9/60 (15%).

### Career:

Farmers: 38/60 (63.4%); Officers: 14/60 (23.3%); Students: 8/60 (13.3%).

### Time of Illness:

Less than 2 years: 6/60 (10%); 2 to 5 years: 15/60 (25%); over 5 years: 39/60 (65%).

### Main Clinical Features:

Signs of the first examination (before taking the drug):
Bone and joint pain: 51/60 (85%); Morning joint stiffness: 49/60 (81.6%); Joint creaking when moving: 9/60 (15%).
The chart of clinical signs after using the drug: (Table 2)

| | Number | Pain level | | | | | | |
|---|---|---|---|---|---|---|---|---|
| | of | Before | 2 | 4 | 6 | 7 | 9 | 12 |
| Symptom | Patients | treatment | weeks | weeks | weeks | weeks | weeks | weeks |
| Pain in many joints | 51 | +++ | ++ | + | 0 | 0 | 0 | 0 |
| Joint stiffness in | 49 | ++ | + | + | 0 | 0 | 0 | 0 |
| mornings | | | | | | | | |
| Creaking sound when | 9 | ++ | + | + | + | 0 | 0 | 0 |
| joints moving | | | | | − | | | |
| Back pain, shoulder | 9 | ++ | + | + | 0 | 0 | 0 | 0 |
| pain | | | | | | | | |
| Sciatica | 3 | +++ | ++ | + | + | 0 | 0 | 0 |
| | | | | | − | | | |
| Numbness, fatigue, | 29 | ++ | + | 0 | 0 | 0 | 0 | 0 |
| sensory disturbances | | | | | | | | |
| in the limbs | | | | | | | | |
| Joint deformity | 3 | + | + | + | + | + | + | + |

* Notes:
A patient may experience many symptoms
Pain level denoted by (+); (+, −): Sometimes it hurts, sometimes it doesn't
* Comments:
After 3 months of continuous use of the drug according to the prescription we found that:

For symptoms of joint pain and stiffness in the morning and back pain, shoulder and neck pain, the improvement is markedly shown after every week of using the drug. After 6 weeks of using the drug, the symptoms of joint pain have completely disappeared (100%). Monitoring the patients for further 6 weeks showed that there is no sign of recurrence;

For symptoms of joint creaking when moving and sciatica, the improvement is markedly shown by each week of using the drug. After 7 weeks of the use, the symptoms of joint pain have completely disappeared. Monitoring the patients for further 5 weeks showed that there is no sign of recurrence;

For symptoms of numbness, fatigue, sensory disturbances in the limbs: the improvement is markedly shown by each week of using the drug. Especially, just after 4 weeks of the use the symptom of joint pain has completely disappeared (100%). Monitoring the patients for further 12 weeks showed that there is no sign of recurrence;

The drug does not work to recover joint deformity, because this is a condition that cannot be treated with oral medications, but often requires surgical intervention.

### Subclinical Symptom: (Table 3)

| | | After treatment | | |
|---|---|---|---|---|
| | | 4 | 6 | 12 |
| Test Names | Before treatment | weeks | weeks | weeks |

| | | | | |
|---|---|---|---|---|
| Blood | White blood | 52/60 (86.6%) | Back to | |
| tests | cells | patients suffered | normal | |
| | (WBCs) | high WBCs | | |
| | Erythrocyte | 56/60 (93.3%) | Back to | |
| | sedimenta- | patients suffered | normal | |
| | tion rate | hight erythrocyte | | |
| | | sedimentation rate | | |
| | Uric acid | 6/60 (10%) | High | Back to |
| | (for Goute | patients suffered | | normal |
| | disease) | high uric acid | | |

| | |
|---|---|
| Glucosa | No change in the use of the drug |

| X-ray | X-ray images shows that bone spurs, |
|---|---|
| | reduced vertebrae height, fibrocartilage, |
| | etc. have not been worsened. |
| | * Comments: |

After 3 months of continuous use of the drug, we found that in addition to very good improvements in the treatment of osteoarthritis, through subclinical tests (according to Table 3) on biochemistry, we also see other very good effects of the drug which are:
After 4 weeks of using the drug, 100% of the patients with high white blood cells returned to normal level;
After 4 weeks of using the drug, 100% of the patients with high erythrocyte sedimentation rate returned to normal level;
After 6 weeks of using the drug, 100% of the patients with high uric acid (Guote disease) returned to normal level;
In addition, the conditions of bone spurs, reduced vertebral height and cartilage fibrosis have stopped not getting worse.
Noted side effects of the drug: (Table 4)

| | Duration | | | | | |
|---|---|---|---|---|---|---|
| Symptom | Week 1 | | Week 2 | Week 4 | Week 8 | Week 12 |
| Allergy | 0/60 | | 0 | 0 | 0 | 0 |
| Nausea | 3/60 | (5%) | 0 | 0 | 0 | 0 |
| Mild digestive | 2/60 | (3.3%) | 0 | 0 | 0 | 0 |
| disorders | | | | | | |
| (diarrhoea) | | | | | | |

* Comments:

As it is known, herbal products are often related to each person's nature, especially in the first time using the drug. The results in Table 4 showed that only 5 patients (a relatively small proportion of 8.3%) had nausea and mild gastrointestinal disturbances in the first week of using the drug. However, from the following weeks those signs were completely gone. Therefore, it can be said that the drug does not cause harmful side effects for patients.

Therefore, it can be confirmed that 100% of the patients are well tolerated after 2 weeks of using the drug.

### Comments

### Regarding the General Characteristics of the Study Group

The study was conducted at Hai Hau District General Hospital, which is a hospital of second class having full modern machinery and equipment and professional doctors who were educated at Hanoi Medical University and have many years of experiences in community treatment;
In this study of 60 patients of all ages, the prevalence of disease was often high in the middle-aged group, and women accounted for almost twice that of men. This rate is also quite similar to the disease model of Vietnam;
Regarding the duration of bone and joint disease, in this study, 65% of the patients have suffered the disease for more than 5 years with typical symptoms of the disease: a lot of pain in bones and joints; Joint stiffness in the morning; Creaking sound when moving, wherein 2 signs of a lot of pain in bones, joints and joint stiffness in the morning are the highest rate (85% and 81.6%, respectively).

### The Effectiveness of the Drug in the Treatment of Osteoarthritis

The effectiveness of reducing the leading sign in osteoarthritis like pain in bones and joints:
After 3 months of using the drug, the patients were examined at 2 weeks, 4 weeks, 6 weeks, 7 weeks, 9 weeks and 12 weeks, and assessed about the level of improvement in pain relief of bone and joint based on the satisfaction of patients. We consider the pain level before treatment to be (+++), then after 2 to 4 weeks of the use this condition was gradually reduced to (++) and (+). Especially after 6 weeks onwards until the end of the 3-month study, the pain in bones and joints was gone and there was no sign of recurrence;
It can be said that after using the drug for 3 consecutive months, bone and joint pain, a condition that causes a lot of depression, interferes with daily activities, reduces quality of life and is the most common condition in the patients with osteoarthritis, 100% cured.

### the Effectiveness of Reducing Morning Joint Stiffness:

Joint stiffness in the morning, after waking up is also a fairly common condition in osteoarthritis patients, it causes a lot of trouble and affects the daily life of the patients;
As shown in Table 2, after treatment the above symptoms in the patients disappeared after 6 weeks of continuous use of the drug.

Thus, after using the drug, morning joint stiffness has been completely eliminated.

The Effectiveness of Reducing the Condition of Joints Creaking when Moving:
Similar to morning stiffness, joint creaking when moving is also a common condition in patients with osteoarthritis, it interferes with patients' activities and work. These are symptoms that patients need to be examined and treated. However, this is a condition that is often overlooked by patients, but over time untreated, can lead to joint deformity;
As shown in Table 2, this situation in terms of speed of improvement is not as fast as the above conditions, however from the 7th week onwards, this situation has also been completely eliminated.

Thus, the drug has the effect of treating joint creaking during movement if patients use it continuously for at least 7 consecutive weeks, and we recommend a use for a minimum of 3 months.

### The Effectiveness of Reducing Conditions of Back Pain, Shoulder Pain:

Back pain and neck pain are also a manifestation of osteoarthritis. And just like the above conditions, back pain and/or shoulder pain appeared in patients with osteoarthritis, after only 6 weeks of using the drug, this condition was completely eliminated and there was no sign of recurrence.

### The Effectiveness of Reducing the Condition of Sciatica:

It seems that the condition of sciatica in the patients is not very specific, with only 3 patients having this symptom in a total of 60 patients studied. These symptoms are common in people with degenerative disc degeneration of the lumbar spine, lumbar spine, etc. This is a difficult condition to treat.

As shown in Table 2, it is not until the 7th week after using the drug that this situation can be reversed significantly.

So it can be said that the drug has the effects of treating sciatica. After 7 to 12 weeks of using the drug, this condition is completely gone.

### The Effectiveness of Reducing the Conditions of Numbness, Fatigue, Sensory Disturbances in Limbs:

Numbness, fatigue, sensory disturbances in the limbs are also a fairly common condition among patients with osteoarthritis, it does not affect life much, but it is quite annoying for patients in daily activities of their life.

It can be affirmed that this is the condition with the fastest improvement rate, after only 4 weeks of using the drug, all the conditions of numbness, fatigue, sensory disturbances in limbs no longer appear and patients get comfortable in life.

### The Side Effects of the Drug:

### for Clinical Manifestations

Through the study on 60 patients, we found that:

100% of the patients are not allergic to the treatment drug. During the first 1 week of product use, 5% of the patients developed nausea and 3% of the patients presented with mild gastrointestinal disturbances such as loose stools, and no patients developed severe diarrhea leading to quitting the treatment.

From the above study, it can be confirmed that the drug does not cause serious side effects for users.

### Test Indexes

All patients had blood tests and X-rays taken before and after treatment.

Regarding blood cells, 52/60 patients had high white blood cells before the treatment. But after only 4 weeks of using the drug, these patients have returned to normal and stable levels;

Regarding erythrocyte sedimentation rate, before the treatment there were 56/60 patients having high erythrocyte sedimentation rate. But after only 4 weeks of using the drug it returned to normal level;

Regarding uric acid content in blood, before the treatment, 6/60 patients had high uric acid content in blood. But test results after 6 weeks of using the drug showed that these patients have uric acid content in the blood returned to normal levels. This is a very good effect for patients with both bone and joint diseases, gout or high uric acid content in the blood;

In addition, when using the drug, the blood sugar content did not change. Therefore, the drug can be used in the treatment of osteoarthritis in diabetic patients.

### Effects of the Present Invention

Clinical trials have shown the effectiveness of the drug for the treatment of osteoarthritis according to the present invention as follows:
Reducing the condition of joint, bone pains;
Reducing the condition of joint stiffness in morning;
Reducing the condition of joint creaking when moving;
Reducing the condition of back pain, shoulder pain;
Reducing the condition of sciatica; and
Reducing the condition of numbness, fatigue, sensory disturbances in limbs; and
Having no side effects in all treated patients.

1. A medicine composition for treatment of bone and joint diseases having ingredients of:
dried extract of the medicinal herbs: 40-75, more preferably 45-70, most preferably 50-65;
tortoiseshell extract: 0.1-20, more preferably 2-18, most preferably 3-15;
mixed bone extract: 0.1-20, more preferably 2-18, most preferably 3-15;
deer antler extract: 0.1-20, more preferably 2-18, most preferably 3-15;
salix alba extract: 5-25, more preferably 7-22, most preferably 10-20;
nattokinase: 0.1-10, more preferably 0.5-8, most preferably 0.7-7;
Na-benzoate: 0-0.5, more preferably 0.05-0.4, most preferably 0.1-0.3,
wherein the herbal medicine mixture comprises the ingredients of: Huang Bai (bark), Liquorice (root/rhizome), Hardy rubber tree (bark), Ox Knee (root), Fang Feng (root), Poria cocos wolf (fruit body), Chinese foxglove (rhizome), Sang Ji Sheng (whole tree), Gentian Root (root), Rhizoma Anemarrhena (rhizome), Peach blossom (blossom), Dried tangerine peel (peel), Chinese peony (root), Dangshen (root), Ginseng (root), Du Huo (root), Dongquai (root), Cinnamon (bough), Xi Xin (root, rhizome), Szechuan lovage (rhizome) and Dwarf umbrella tree (root).

2. The medicine composition according to claim 1, wherein the herbal medicine mixture has the following proportions of ingredients (% by weight):
Huang Bai: 10-45; Liquorice: 0.1-10; Hardy rubber tree: 0.5-15; Ox Knee: 0.1-10; Fang Feng: 0.1-10; Poria cocos wolf: 0.5-15; Chinese foxglove: 0.5-15; Sang Ji Sheng: 0.1-10; Gentian Root: 0.1-10; Rhizoma Anemarrhena: 0.1-10; Peach blossom: 0.5-15; Dried tangerine peel: 0.5-15; Chinese peony: 1-25; Dangshen: 1-15; Ginseng: 1-15; Du Huo: 0.1-10; Dongquai: 1-15; Cinnamon: 0.1-10; Xi Xin: 0.1-5; Szechuan lovage: 0.1-10 and Dwarf umbrella tree 0.1-10.

3. The medicine composition according to claim 1, wherein the herbal medicine mixture preferably has the following proportions of ingredients (% by weight):
Huang Bai: 15-40; Liquorice: 0.5-8; Hardy rubber tree: 0.7-12; Ox Knee: 0.5-8; Fang Feng: 0.5-8; Poria cocos wolf: 0.7-12; Chinese foxglove: 0.7-12; Sang Ji Sheng: 0.5-8; Gentian Root: 0.5-8; Rhizoma Anemarrhena: 0.5-8; Peach blossom: 0.7-12; Dried tangerine peel: 0.7-12; Chinese peony: 5-20; Dangshen: 1.5-12; Ginseng: 1.5-12, Du Huo: 0.5-8; Dongquai: 1.5-12; Cinnamon: 0.5-8; Xi Xin: 0.2-4; Szechuan lovage: 0.5-8 and Dwarf umbrella tree 0.5-8.

4. The medicine composition according to claim 1, wherein the herbal medicine mixture more preferably has the following proportions of ingredients (% by weight):
Huang Bai: 20-35; Liquorice: 0.7-5; Hardy rubber tree: 1-10; Ox Knee: 0.7-5; Fang Feng: 0.7-5; Poria cocos wolf: 1-10; Chinese foxglove: 1-10; Sang Ji Sheng: 0.7-5; Gentian Root: 0.7-7; Rhizoma Anemarrhena: 0.7-7; Peach blossom: 1-10; Dried tangerine peel: 1-10; Chinese peony: 8-18; Dangshen: 2-10; Ginseng: 2-10; Du Huo: 0.7-7; Dongquai: 2-10; Cinnamon: 0.7-5; Xi Xin: 0.3-3; Szechuan lovage: 0.7-7 and Dwarf umbrella tree 0.7-7.

5. A process for producing medicine composition for treatment of bone and joint diseases according to claim 1 including the following steps:
i) Preparing medicinal herbs of: Huang Bai (bark), Liquorice (root/rhizome), Hardy rubber tree (bark), Ox Knee (root), Fang Feng (root), Poria cocos wolf (fruit body), Chinese foxglove (rhizome), Sang Ji Sheng (whole tree), Gentian Root (root), Rhizoma Anemarrhena (rhizome), Peach blossom (blossom), Dried tangerine peel (peel), Chinese peony (root), Dangshen (root), Ginseng (root) Du Huo (root), Dongquai (root), Cinnamon (bough), Xi Xin (root, rhizome), Szechuan lovage (rhizome) and Dwarf umbrella tree (root), mixed in a predefined proportion, washed, dried, then stirred in a hot pan;
ii) Extracting the herbal medicine mixture to obtain a dried extract of the herbal medicine mixture;
iii) Preparating mixed the bone extract, the tortoiseshell extract and the deer antler extract:
washing animal bones, tortoiseshells, deer antlers from trash and tissues, then incubating them with 10% ethanol 45% for 2 hours, then feeding them into a boiling pot, periodically extracting the boiled extract every 24 hours for 3 times, during said process boiling water is added to keep the boiled material under the water, to obtain mixed extracts;
evaporating the mixed extracts to a moisture content of 20-25%, then adding 0.5% preservatives nipagin/nipasol at a ratio of 9/1 or sodium benzoate dissolved in ethanol 70%, stirring the mixture to evenly distributed state, then evaporating evenly distributed mixture to a moisture content of about 25%;
if the batches of extracts are took place continuously, the third extraction can be used as a solvent for the first extraction of the following batch; and
iv) Obtaining the medicine compositions for treatment of bone and joint diseases by mixing the semi-products obtained from the above said steps at the following ration (% by weight):
dried extract of the medicinal herbs: 40-75, more preferably 45-70, most preferably 50-65;
tortoiseshell extract: 0.1-20, more preferably 2-18, most preferably 3-15;
mixed bone extract: 0.1-20, more preferably 2-18, most preferably 3-15;
deer antler extract: 0.1-20, more preferably 2-18, most preferably 3-15;
salix alba extract: 5-25, more preferably 7-22, most preferably 10-20;
nattokinase: 0.1-10, more preferably 0.5-8, most preferably 0.7-7;
Na-benzoate: 0-0.5, more preferably 0.05-0.4, most preferably 0.1-0.3.

6. The process according to claim 5, wherein the step of preparing the medicinal herbs for producing solid extracts consists of grinding a mixture of the medicinal herbs into a powder, then sieving through a sieve to separate them into particles with a particle size of <0.2 mm (fine extract powder) and those with a particle size >0.2 mm (crude powder).

7. The process according to claim 6, wherein the step extracting mixture the medicine composition consists of preparating binders is carried out as follows:
preparating extract mixture of the medicinal herbs by washing the medicinal herbs and then chopping them, then in combination with the crude powder having a particle size of >0.2 mm which has been feeded into damp cloth bags and sealed;
putting the bags of the crude powder and the chopped medicinal herbs into an extraction pot, adding water to about 20 cm above the surface of the medicinal herbs, heating up to boiling point and keeping boiling for about 3 hours, then collecting an extract;
repeating the extraction process 3 times, combining the extracts and filtering them through a sieve of size 0.2 mm, concentrating semi-solid extracts by direct evaporation to about 30% moisture content;
dissolving the bone extract obtained in step (iii) into boiling water, adding said semi-solid extracts mixture of the medicinal herbs with 30% moisture content and heating up until evenly dispersed into a homogeneous mixture (Mixture A) to be used for making the binders;
preparating NaCMC binder as follows:
weighing the purified water, adding nipagin, nipasol into the water then stirring until completely dissolved state, slowly adding NaCMC while stirring the solution to evenly distributed state, letting the solution swell for about 8-12 hours to obtain a NaCMC binder;
preparating starch binder as follows:
mixing purified water, amidon, sodium benzoate, cassava starch in an appropriate proportion, well stirring the mixture while heating it up to form the starch binder;
mixing the binders as follows:
thoroughly mixing the completely swelled NaCMC binder with the starch binder and extract mixture (A) when they are still hot while well stirring to obtain the binder mixture that is used for producing the medicine composition.

8. The process according to claim 5, wherein the step extracting the herbal medicine mixture to obtain the medicinal herbs extracts for producing hard capsules is carried out by two stages as follows:
by supercritical CO2 extraction:
washing the medicinal herbs, feeding them into extraction pot for supercritical CO2 extraction in 2 repeated cycles, each cycle is extracted with co-solvent EtOH/CO2 0-5% (weight) under the following conditions: pressure 20-40 MPas, temperature 40-70° C., duration 60-180 minutes, CO2 flow 100-500 kg/h; after that collecting the extracts of the 2 cycles then packing them into 2-layer PE bags or aluminum bags; the residues of said supercritical CO2 extraction will be used for further water extraction;
by water extraction:
pouring water over 10-15 cm above the surface of the residue and boiling them for 3 hours of boiling, then collecting the first extract and adding water to boil for the second time, repeating the extraction two times; combining the extracts, filtering through a coarse cloth and concentrating the extracts until obtaining the extracts with a moisture content of about 20%; adding nipagin and nipasol or sodium benzoate dissolved in ethanol 96%, stirring well; casting into extract cakes to obtain a concentrated extract a moisture content of 20%;
combining the concentrated extract of 20% moisture content and the extract of supercritical CO2 extraction to form a mixture of the concentrated medicinal herbs that is dried by a static oven or spray-dried, at a high temperature of about 85° C. to dry, letting it cool and then grind it finely with a hammer mill and sieve it through a 0.2 mm sieve to obtain a mixture of powdered extract of the medicinal herbs used to prepare preparations in the following stage.

9. The process according to claim 5, wherein the herbal medicine mixture has the following proportions of ingredients (% by weight):
Huang Bai: 10-45; Liquorice: 0.1-10; Hardy rubber tree: 0.5-15; Ox Knee: 0.1-10; Fang Feng: 0.1-10; Poria cocos wolf: 0.5-15; Chinese foxglove: 0.5-15; Sang Ji Sheng: 0.1-10; Gentian Root: 0.1-10; Rhizoma Anemarrhena: 0.1-10; Peach blossom: 0.5-15; Dried tangerine peel: 0.5-15; Chinese peony: 1-25; Dangshen: 1-15; Ginseng: 1-15; Du Huo: 0.1-10; Dongquai: 1-15; Cinnamon: 0.1-10; Xi Xin: 0.1-5; Szechuan lovage: 0.1-10 and Dwarf umbrella tree 0.1-10.

10. The process according to claim 5, wherein more preferably the herbal medicine mixture has the following proportions of ingredients (% by weight):
Huang Bai: 15-40; Liquorice: 0.5-8; Hardy rubber tree: 0.7-12; Ox Knee: 0.5-8; Fang Feng: 0.5-8; Poria cocos wolf: 0.7-12; Chinese foxglove: 0.7-12; Sang Ji Sheng: 0.5-8; Gentian Root: 0.5-8; Rhizoma Anemarrhena: 0.5-8; Peach blossom: 0.7-12; Dried tangerine peel: 0.7-12; Chinese peony: 5-20; Dangshen: 1.5-12; Dangshen: 1.5-12; Du Huo: 0.5-8; Dongquai: 1.5-12; Cinnamon: 0.5-8; Xi Xin: 0.2-4; Szechuan lovage: 0.5-8 and Dwarf umbrella tree 0.5-8.

11. The process according to claim 5, wherein most preferably the herbal medicine mixture has the following proportions of ingredients (% by weight):
Huang Bai: 20-35; Liquorice: 0.7-5; Hardy rubber tree: 1-10; Ox Knee: 0.7-5; Fang Feng: 0.7-5; Poria cocos wolf: 1-10; Chinese foxglove: 1-10; Sang Ji Sheng: 0.7-5; Gentian Root: 0.7-7; Rhizoma Anemarrhena: 0.7-7; Peach blossom: 1-10; Dried tangerine peel: 1-10; Chinese peony: 8-18; Dangshen: 2-10; Ginseng: 2-10; Du Huo: 0.7-7; Dongquai: 2-10; Cinnamon: 0.7-5; Xi Xin: 0.3-3; Szechuan lovage: 0.7-7 and Dwarf umbrella tree 0.7-7.

12. The process according to claim 5, wherein to prepare the medicine composition in the form of solid extracts, the preparation step is carried out as follows:
loading the prepared fine extract powders into a mixer, mixing the powders for about 10 to 35 minutes for obtaining a homogeneous dry mixture of extract powders;
slowly adding all binders to the obtained dry mixture of extract powders, mixing for about 5 to 15 minutes for obtaining a homogeneous wet mass of the extracts;
dividing the obtained wet mass of the extracts by a pelletizing machine;
sieve to classify the obtained extract pellets to obtain extract pellets of size 4-6.0 mm;
drying the obtained extract pellets obtained at a temperature of 65-90° C. to a moisture content of not over 10% to obtain dry extract pellets;
then sorting the obtained dry extract pellets by a pellet sorting machine, collecting the extract pellets with the required size.

13. The process according to claim 1, wherein to prepare the medicine composition in the form of hard capsules, the preparation step is carried out as follows:
thoroughly mixing fermented soybean paste (nattokinase), dry powder of the medicinal herbs and powders of bone extract and canxi carbonat according to the principle of geometric dilution;
thoroughly mixing cassava starch with the obtained mixture according to the principle of geometric dilution, finally with a mixture of glidants consisting of talc powder or erosil and magnesi stearat (already mixed) to obtain a powdered mixture for encapsulation;
encapsulating the powdered mixture by an automatic encapsulation machine to obtain capsules with the encapsulation powder weight of about 0.786 g to 0.913 g (0.85 g±7.5%).

14. A process for producing medicine composition for treatment of bone and joint diseases according to claim 2 including the following steps:
i) Preparing medicinal herbs of: Huang Bai (bark), Liquorice (root/rhizome), Hardy rubber tree (bark), Ox Knee (root), Fang Feng (root), Poria cocos wolf (fruit body), Chinese foxglove (rhizome), Sang Ji Sheng (whole tree), Gentian Root (root), Rhizoma Anemarrhena (rhizome), Peach blossom (blossom), Dried tangerine peel (peel), Chinese peony (root), Dangshen (root), Ginseng (root) Du Huo (root), Dongquai (root), Cinnamon (bough), Xi Xin (root, rhizome), Szechuan lovage (rhizome) and Dwarf umbrella tree (root), mixed in a predefined proportion, washed, dried, then stirred in a hot pan;
ii) Extracting the herbal medicine mixture to obtain a dried extract of the herbal medicine mixture;
iii) Preparating mixed the bone extract, the tortoiseshell extract and the deer antler extract:
washing animal bones, tortoiseshells, deer antlers from trash and tissues, then incubating them with 10% ethanol 45% for 2 hours, then feeding them into a boiling pot, periodically extracting the boiled extract every 24 hours for 3 times, during said process boiling water is added to keep the boiled material under the water, to obtain mixed extracts;
evaporating the mixed extracts to a moisture content of 20-25%, then adding 0.5% preservatives nipagin/nipasol at a ratio of 9/1 or sodium benzoate dissolved in ethanol 70%, stirring the mixture to evenly distributed state, then evaporating evenly distributed mixture to a moisture content of about 25%;
if the batches of extracts are took place continuously, the third extraction can be used as a solvent for the first extraction of the following batch; and
iv) Obtaining the medicine compositions for treatment of bone and joint diseases by mixing the semi-products obtained from the above said steps at the following ration (% by weight):
dried extract of the medicinal herbs: 40-75, more preferably 45-70, most preferably 50-65;
tortoiseshell extract: 0.1-20, more preferably 2-18, most preferably 3-15;
mixed bone extract: 0.1-20, more preferably 2-18, most preferably 3-15;
deer antler extract: 0.1-20, more preferably 2-18, most preferably 3-15;
salix alba extract: 5-25, more preferably 7-22, most preferably 10-20;
nattokinase: 0.1-10, more preferably 0.5-8, most preferably 0.7-7;
Na-benzoate: 0-0.5, more preferably 0.05-0.4, most preferably 0.1-0.3.

15. A process for producing medicine composition for treatment of bone and joint diseases according to claim 3 including the following steps:
i) Preparing medicinal herbs of: Huang Bai (bark), Liquorice (root/rhizome), Hardy rubber tree (bark), Ox Knee (root), Fang Feng (root), Poria cocos wolf (fruit body), Chinese foxglove (rhizome), Sang Ji Sheng (whole tree), Gentian Root (root), Rhizoma Anemarrhena (rhizome), Peach blossom (blossom), Dried tangerine peel (peel), Chinese peony (root), Dangshen (root), Ginseng (root) Du Huo (root), Dongquai (root), Cinnamon (bough), Xi Xin (root, rhizome), Szechuan lovage (rhizome) and Dwarf umbrella tree (root), mixed in a predefined proportion, washed, dried, then stirred in a hot pan;
ii) Extracting the herbal medicine mixture to obtain a dried extract of the herbal medicine mixture;
iii) Preparating mixed the bone extract, the tortoiseshell extract and the deer antler extract:
washing animal bones, tortoiseshells, deer antlers from trash and tissues, then incubating them with 10% ethanol 45% for 2 hours, then feeding them into a boiling pot, periodically extracting the boiled extract every 24 hours for 3 times, during said process boiling water is added to keep the boiled material under the water, to obtain mixed extracts;
evaporating the mixed extracts to a moisture content of 20-25%, then adding 0.5% preservatives nipagin/nipasol at a ratio of 9/1 or sodium benzoate dissolved in ethanol 70%, stirring the mixture to evenly distributed state, then evaporating evenly distributed mixture to a moisture content of about 25%;
if the batches of extracts are took place continuously, the third extraction can be used as a solvent for the first extraction of the following batch; and
iv) Obtaining the medicine compositions for treatment of bone and joint diseases by mixing the semi-products obtained from the above said steps at the following ration (% by weight):
dried extract of the medicinal herbs: 40-75, more preferably 45-70, most preferably 50-65;
tortoiseshell extract: 0.1-20, more preferably 2-18, most preferably 3-15;
mixed bone extract: 0.1-20, more preferably 2-18, most preferably 3-15;
deer antler extract: 0.1-20, more preferably 2-18, most preferably 3-15;
salix alba extract: 5-25, more preferably 7-22, most preferably 10-20;
nattokinase: 0.1-10, more preferably 0.5-8, most preferably 0.7-7;
Na-benzoate: 0-0.5, more preferably 0.05-0.4, most preferably 0.1-0.3.

16. A process for producing medicine composition for treatment of bone and joint diseases according to claim 4 including the following steps:
i) Preparing medicinal herbs of: Huang Bai (bark), Liquorice (root/rhizome), Hardy rubber tree (bark), Ox Knee (root), Fang Feng (root), Poria cocos wolf (fruit body), Chinese foxglove (rhizome), Sang Ji Sheng (whole tree), Gentian Root (root), Rhizoma Anemarrhena (rhizome), Peach blossom (blossom), Dried tangerine peel (peel), Chinese peony (root), Dangshen (root), Ginseng (root) Du Huo (root), Dongquai (root), Cinnamon (bough), Xi Xin (root, rhizome), Szechuan lovage (rhizome) and Dwarf umbrella tree (root), mixed in a predefined proportion, washed, dried, then stirred in a hot pan;
ii) Extracting the herbal medicine mixture to obtain a dried extract of the herbal medicine mixture;
iii) Preparating mixed the bone extract, the tortoiseshell extract and the deer antler extract:
washing animal bones, tortoiseshells, deer antlers from trash and tissues, then incubating them with 10% ethanol 45% for 2 hours, then feeding them into a boiling pot, periodically extracting the boiled extract every 24 hours for 3 times, during said process boiling water is added to keep the boiled material under the water, to obtain mixed extracts;
evaporating the mixed extracts to a moisture content of 20-25%, then adding 0.5% preservatives nipagin/nipasol at a ratio of 9/1 or sodium benzoate dissolved in ethanol 70%, stirring the mixture to evenly distributed state, then evaporating evenly distributed mixture to a moisture content of about 25%;
if the batches of extracts are took place continuously, the third extraction can be used as a solvent for the first extraction of the following batch; and
iv) Obtaining the medicine compositions for treatment of bone and joint diseases by mixing the semi-products obtained from the above said steps at the following ration (% by weight):
dried extract of the medicinal herbs: 40-75, more preferably 45-70, most preferably 50-65;
tortoiseshell extract: 0.1-20, more preferably 2-18, most preferably 3-15;
mixed bone extract: 0.1-20, more preferably 2-18, most preferably 3-15;
deer antler extract: 0.1-20, more preferably 2-18, most preferably 3-15;
salix alba extract: 5-25, more preferably 7-22, most preferably 10-20;
nattokinase: 0.1-10, more preferably 0.5-8, most preferably 0.7-7;
Na-benzoate: 0-0.5, more preferably 0.05-0.4, most preferably 0.1-0.3.
